(19) United States
(12) Patent Application Publication
(10) Pub. No.: US 2009/0062301 A1
(43) Pub. Date: Mar. 5, 2009

(54) PHARMACEUTICAL COMBINATION COMPOSITION COMPRISING AT LEAST ONE PKC INHIBITOR AND AT LEAST ONE JA K3 KINASE INHIBITOR FOR TREATING AUTOIMMUNE DISORDERS

(76) Inventor: Axel Maibucher, Allschwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/282,416

(22) PCT Filed: Mar. 19, 2007
(86) PCT No.: PCT/EP2007/002416

§ 371 Date: Sep. 10, 2008

(30) Foreign Application Priority Data
Mar. 21, 2006 (GB) 0605691.5

(51) Int. Cl. A61K 31/496; A61K 31/4725; A61K 31/4545; A61K 31/454; A61K 31/519; A61P 37/06

(52) U.S. Cl. 514/252.17; 514/307; 514/318; 514/323; 514/265.1

(57) ABSTRACT

The present invention relates to a pharmaceutical combination comprising at least one PKC inhibitor, in particular indolylmaleimide derivatives, and at least one JAK3 kinase inhibitor and the uses of such a combination e.g. in autoimmune diseases, e.g. in preventing or treating type I diabetes mellitus and disorders associated therewith, or in transplantation.

The present invention relates to a pharmaceutical combination comprising at least one PKC inhibitor, in particular indolylmaleimide derivatives, and at least one JAK3 kinase inhibitor and the uses of such a combination e.g. in autoimmune diseases, e.g. in preventing or treating type I diabetes mellitus and disorders associated therewith, or in transplantation.

The present invention further relates to a pharmaceutical combination comprising at least one and at least one JAK3 kinase inhibitor and the uses of such a combination e.g. in autoimmune diseases, e.g. in preventing or treating type I diabetes mellitus and disorders associated therewith, or in transplantation.

In spite of numerous treatment options for organ transplant and autoimmune disease patients, there remains a need for effective and safe immunosuppressive agents and a need for their preferential use in combination therapy.

It has now been found that a combination comprising at least one PKC inhibitor and a Janus Kinase 3 (JAK3) kinase inhibitor, e.g. as defined below, has a beneficial effect on autoimmune diseases, e.g. type I diabetes and the disorders associated therewith, or graft rejection.

The PKC inhibitors of the invention may be staurosporine analogues or maleimide derivatives. For example, they may be of formula I

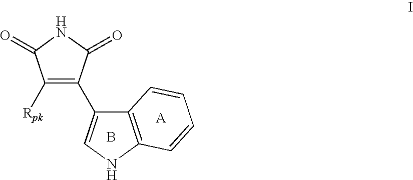

wherein Rₚₖ is an aromatic cycle, e.g. an aromatic heterocycle, optionally fused to another cycle, e.g. another aromatic cycle, optionally an aromatic heterocycle; Rₚₖ and the fused cycle being optionally substituted; and the cycles A and B being optionally substituted.

Examples of appropriate PKC inhibitors are, for example:
Compounds as disclosed in EP1337527A1, and EP 1490355A1, e.g. a compound of formula II

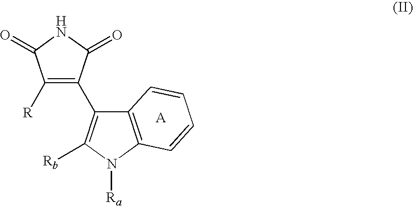

wherein

Rₐ is H; C₁₋₄alkyl; or C₁₋₄alkyl substituted by OH, NH₂, NHC₁₋₄alkyl or N(di-C₁₋₄alkyl)₂;

R_{b} is H; or C₁₋₄alkyl; and

R is a radical of formula (a), (b), (c), (d), (e) or (f)

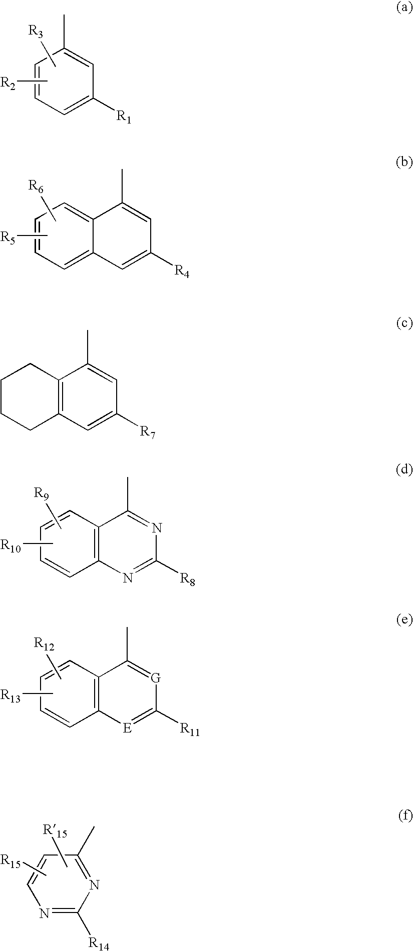

wherein
each of R₁, R₄, R₇, R₈, R₁₁ and R₁₄ is OH; SH; a heterocyclic residue; NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α

—X—R_{c}—Y (α)
wherein X is a direct bond, O, S or NR₁₈ wherein R₁₈ is H or C₁₋₄alkyl,
R_{c} is C₁₋₄alkylene or C₁₋₄alkylene wherein one CH₂ is replaced by CRₓR_{y} wherein one of Rₓ and R_{y} is H and the other is CH₃ each of Rₓ, and R_{y} is CH₃ or Rₓ and R_{y} form together —CH₂—CH₂—, and
Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₉ and R₂₀ form together with the nitrogen atom to which they are bound a heterocyclic residue;
each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;
either E is —N═ and G is —CH═ or E is —CH═ and G is —N═; and

ring A is optionally substituted,

or a pharmaceutically acceptable salt or hydrate thereof.
Compounds of formula (III)

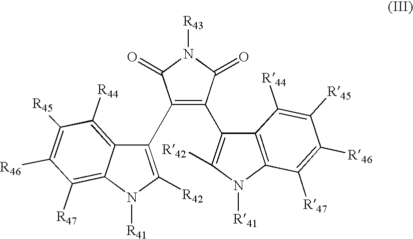

wherein

R₄₁ is a group of formula (g), (h) or (i)

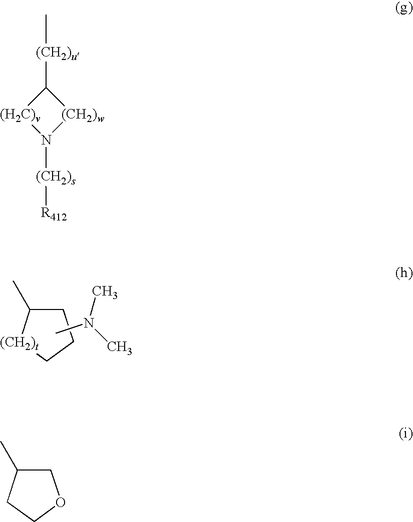

wherein each of v and w independently is 1, 2, 3, or 4;
s is 0, 1, 2 or 3;
t is 1 or 2;
u is 0 or 1; and
R₄₁₂ is hydrogen, alkyl, haloalkyl, cycloalkyl, acetyl, aryl, —CH(aryl)₂, amino, monoalkylamino, dialkylamino, guanidino, —C(═N(alkoxycarbonyl))NH(alkyoxycarbonyl), amidino, hydroxy, carboxy, alkoxycarbonyl or heterocyclyl;

R′₄₁ is hydrogen, C₁₋₄alkyl, aminoalkyl, monoalkylaminoalkyl, or dialkylaminoalkyl,

each of R₄₂ and R′₄₂, independently, is hydrogen, alkyl, alkoxyalkyl, hydroxyalkyl, C₁-C₃alkylthio, S(O)C₁-C₃alkyl, CF₃;

R₄₃ is hydrogen or CH₃CO—; and

each of R₄₄, R′₄₄, R₄₅, R′₄₅, R₄₆, R′₄₆, R₄₇ and R′₄₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl,

or a pharmaceutically acceptable salt, hydrate or solvate thereof.

The compounds of formula (I), (II) and (III) may be synthesized as known in the art, e.g. as described in U.S. Pat. No. 6,645,970 or EP1490355A1 (for compounds of formula II), EP1490355A1 (for compounds of formula II), U.S. Pat. No. 5,545,636 (for compounds of formula II).

The PKC inhibitors of the invention may inhibit several isoforms of the PKC, in particular they may selectively inhibit specific PKC isoforms, i.e. be selective PKC inhibitors, i.e. isozyme-selective PKC inhibitors. Preferably, the PKC inhibitors of the invention are able to selectively inhibit PKC isoforms which are selected from the classical PKC isoforms (α, β₁, β₂, γ) and novel PKC isoforms (ε, η, δ, θ), more preferably selected from the α, β (β₁ and β₂ isoforms) and θ PKC isoforms. Preferred PKC inhibitors of the invention are able to selectively inhibit the α, β, and optionally θ, isoforms of PKC.

For example, the PKC inhibitor of the invention may possess a selectivity for one or more PKC isoforms, e.g. PKC alpha or PKC alpha, beta and optionally theta, over the other PKC isoforms of at least 20 fold, e.g. 100, 500, 1000 or 2000 fold.

The PKC inhibition activity of the PKC inhibitors of the invention may be determined in an Allogeneic Mixed Lymphocyte Reaction (MLR) assay. MLR assay can be done according to known methods, e.g. mouse of human MLR assay, e.g. as disclosed in EP1337527A1, the content regarding the MLR assay being incorporated herein by reference.

In a preferred embodiment, the PKC inhibitors of the invention show an IC₅₀ value, e.g. for the α and β, and optionally θ, PKC isoforms, of 1 μM or less, preferably 10 nM or less in the hereinabove mentioned assay.

In formula II, any alkyl or alkyl moiety in e.g. alkoxy may be linear or branched. Halogen may be F, Cl, Br or I, preferably F or Cl. Any aryl may be phenyl or naphthyl, preferably phenyl.

By heterocyclic residue as Rₚₖ, R₁, R₄, R₇, R₈, R₁₁, R₁₄ or Y or formed, respectively, by NR₁₆R₁₇ or NR₁₉R₂₀, is meant a three to eight, preferably five to eight, membered saturated, unsaturated or aromatic heterocyclic ring comprising 1 or 2 heteroatoms, preferably selected from N, O and S, and optionally substituted.

Suitable examples of heterocyclic residue as R₁, R₄, R₇, R₈, R₁₁, R₁₄ or Y or formed, respectively, by NR₁₆R₁₇ or NR₁₉R₂₀, include e.g. pyridyl, e.g. 3- or 4-pyridyl, piperidyl, e.g. piperidin-1-yl, 3- or 4-piperidyl, homopiperidyl, piperazinyl, e.g. 1-piperazinyl, homopiperazinyl, morpholin-4-yl, imidazolyl, imidazolidinyl, pyrrolyl or pyrrolidinyl, optionally substituted, e.g. mono- or polysubstituted.

Suitable examples of heterocyclic residue as R₁₁ include e.g. 4,7-diaza-spiro[2.5]oct-7-yl.

When the heterocyclic residue is substituted, this may be on one or more ring carbon atoms and/or on a ring nitrogen atom when present. Examples of a substituent on a ring carbon atom include e.g. C₁₋₄alkyl e.g. CH₃;

C₃₋₆cycloalkyl e.g. cyclopropyl, optionally further substituted by C₁₋₄alkyl;

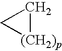

wherein p is 1, 2 or 3, preferably 1; CF₃; halogen; OH; NH₂; —CH₂—NH₂; —CH₂—OH; piperidin-1-yl; or pyrrolidinyl. Examples of a substituent on a ring nitrogen atom are e.g. C₁₋₆alkyl; acyl, e.g. R′ₓ—CO wherein R′ₓ is H, C₁₋₆alkyl or phenyl optionally substituted by C₁₋₄alkyl, C₁₋₄alkoxy or amino, e.g formyl; C₃₋₆cycloalkyl; C₃₋₆cycloalkyl-C₁₋₄alkyl; phenyl; phenyl-C₁₋₄alkyl e.g. benzyl; a heterocyclic residue, e.g. as disclosed above, e.g. an aromatic heterocyclic residue comprising 1 or 2 nitrogen atoms; or a residue of formula β

—R₂₁—Y′ (β)

wherein R₂₁ is C₁₋₄alkylene or C₂₋₄alkylene interrupted by O and Y′ is OH, NH₂, NH(C₁₋₄alkyl) or N(C₁₋₄alkyl)₂.

In formula II C₂₋₄alkylene interrupted by O may be e.g. —CH₂—CH₂—O—CH₂—CH₂—.

In formula II, when the substituent on a cyclic nitrogen is a heterocyclic residue, it may be a five or six membered saturated, unsaturated or aromatic heterocyclic ring comprising 1 or 2 heteroatoms, preferably selected from N, O and S. Examples include e.g. 3- or 4-pyridyl, piperidyl, e.g. piperidin-1-yl, 3- or 4-piperidyl, homopiperidyl, piperazinyl, homopiperazinyl, pyrimidinyl, morpholin-4-yl, imidazolyl, imidazolidinyl, pyrrolyl or pyrrolidinyl,

In formula II, when Rₐ is substituted C₁₋₄alkyl, the substituent is preferably on the terminal carbon atom.

When ring A is substituted, it may be mono- or polysubstituted, preferably monosubstituted, the substituent(s) being selected from the group consisting of e.g. halogen, OH, C₁₋₄alkoxy, e.g. OCH₃, C₁₋₄alkyl, e.g. CH₃, NO₂, CF₃, NH₂, NHC₁₋₄alkyl, N(di-C₁₋₄alkyl)₂ and CN. For example, ring A may be a residue of formula

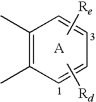

wherein

R_{d} is H; C₁₋₄alkyl; or halogen; and

Rₑ is OH; NO₂; NH₂; NHC₁₋₄alkyl; or N(di-C₁₋₄alkyl)₂.

Preferably R_{d} is in position 1; preferably Rₑ is in position 3.

When R_{c} has a CH₂ replaced by CRₓR_{y}, it is preferably the CH₂ bearing Y.

Examples of heterocyclic residue as R₁, R₄, R₇, R₈, R₁₁, R₁₄ or Y or formed, respectively, by NR₁₆R₁₇ or NR₁₉R₂₀, include e.g. a residue of formula (γ)

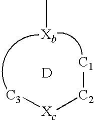

wherein

the ring D is a 5, 6 or 7 membered saturated, unsaturated or aromatic ring;
X_{b} is —N—, —C═ or —CH—;
X_{c} is —N═, —NR_{f}—, —CR′_{f}═ or —CHR′_{f}— wherein R_{f} is a substituent as indicated above for a ring nitrogen atom, and R′_{f} is a substituent as indicated above for a ring carbon atom;
the bond between C₁ and C₂ is either saturated or unsaturated;
each of C₁ and C₂, independently, is a carbon atom which is optionally substituted by one or two substituents selected among those indicated above for a ring carbon atom; and
the line between C₃ and X_{b} and between C₁ and X_{b}, respectively, represents the number of carbon atoms as required to obtain a 5, 6 or 7 membered ring D.

A preferred residue of formula (γ) is one wherein the ring D forms a 1,4-piperazinyl ring optionally C- and/or N-substituted as indicated.

Representative examples of a residue of formula (γ) are e.g. 3- or 4-pyridyl; piperidin-1-yl; 1-N—(C₁₋₄alkyl)- or -(ω-hydroxy-C₁₋₄alkyl)-3-piperidyl; morpholin-4-yl; imidazolyl; pyrrolidinyl; 1-piperazinyl; 2-C₁₋₄alkyl- or —C₃₋₆cycloalkyl-1-piperazinyl; 3-C₁₋₄alkyl- or —C₃₋₆cycloalkyl-1-piperazinyl; 2,2- or 3,5- or 2,5- or 2,6-di(C₁₋₄alkyl)-1-piperazinyl; 3,4,5-tri-(C₁₋₄alkyl)-1-piperazinyl; 4-N—(C₁₋₄alkyl)- or -(ω-hydroxy-C₁₋₄alkyl)- or -(ω-dimethylamino-C₁₋₄alkyl)-1-piperazinyl; 4-N-pyridin-4-yl-1-piperazinyl; 4-N-phenyl- or —C₃₋₆cycloalkyl-1-piperazinyl; 4-N—(C₁₋₄alkyl)- or -(ω-hydroxy-C₁₋₄alkyl)-3-C₁₋₄alkyl- or -3,3-di(C₁₋₄alkyl)-1-piperazinyl; 4-N-(1-C₁₋₄alkyl-C₃₋₆cycloalkyl)-1-piperazinyl; 4-N-formyl-1-piperazinyl; 4-N-pyrimidin-2-yl-1-piperazinyl; 4,7-diaza-spiro[2.5]oct-7-yl or 4-N—C₁₋₄alkyl-1-homopiperazinyl.

The compounds of formulae I and II may exist in free form or in salt form, e.g. addition salts with e.g. organic or inorganic acids, for example, hydrochloric acid, acetic acid, when R₁, R₄, R₇, R₈, R₁₁ or R₁₄ and/or R₂, R₃, R₅, R₆, R₉, R₁₀, R₁₂, R₁₃ or R₁₅ comprises an optionally substituted amino group or a heterocyclic residue which can form acid addition salts.

It will be appreciated that the compounds of formula I, formula II and formula III may exist in the form of optical isomers, racemates or diastereoisomers. For example, a ring carbon atom bearing a substituent in the heterocyclic residue as R₁, R₄, R₇, R₈, R₁₁, R₁₄ or Y or formed, respectively, by NR₁₆R₁₇ or NR₁₉R₂₀, is asymmetric and may have the D- or L-configuration. It is to be understood that the present invention embraces all enantiomers and their mixtures. Similar considerations apply in relation to starting materials exhibiting asymetric carbon atoms as mentioned.

In the compounds of formula II, the following significances are preferred individually or in any sub-combination:
1. Rₐ is H or CH₃;
2. R_{b} is H;
3. Ring A is unsubstituted; or is substituted by methyl in position 7;
4. Preferred heterocyclic residue as formed by NR₁₆R₁₇ is e.g. piperazin-1-yl optionally N-substituted, e.g. by C₁₋₄alkyl, ω-hydroxy-C₁₋₄alkyl, ω-dimethylamino-C₁₋₄alkyl, C₅₋₆cycloalkyl, C₁₋₄alkyl-C₅₋₆cycloalkyl, an aromatic heterocyclic residue comprising 1 or 2 nitrogen atoms, e.g. pyridyl or pyrimidin-2-yl, or a residue of formula β as defined above and/or optionally C-substituted, e.g. by CH₃ e.g. in positions 2, and/or 3 and/or 5 and/or 6 and/or 2, 2 or 3,3 or by

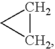

e.g. in position 2 or 3; piperidin-1-yl optionally C-substituted, e.g. in position 4, by NH₂, —CH₂—NH₂ or piperidin-1-yl, or in position 3, e.g. by OH or NH₂; or pyrrolidinyl optionally C-substituted in position 3 by OH or NH₂;
5. R₁₈ is H or CH₃;
6. R_{c} is C₁₋₄alkylene or C₁₋₄alkylene wherein the terminal CH₂ is replaced by CRₓR_{y} wherein Rₓ and R_{y} form together —CH₂—CH₂—;
7. X is O;
8. The radical of formula (α) is —O—CH₂—CH₂—Y;
9. Each of R₁₉ and R₂₀ is H, C₁₋₄alkyl, e.g. methyl, C₁₋₄alkyl substituted on the terminal carbon atom by OH, e.g. —CH₂—CH₂—OH, or cyclopropyl;
10. Preferred heterocyclic residue as formed by NR₁₉R₂₀ is e.g. piperazin-1-yl optionally N-substituted by C₁₋₄alkyl or a residue of formula β; piperidin-1-yl; 1-(C₁₋₄alkyl)-piperidin-3-yl; 3- or 4-pyridyl; imidazolyl; pyrrolidinyl; or morpholin-4-yl;
11. Each of R₁, R₄, R₇, R₈, R₁₁, or R₁₄, independently, is 1-N-methyl-piperidin-4-yl; 4-methyl-piperazin-1-yl; 4-methyl-1-homopiperazinyl; 4-(2-hydroxyethyl)-piperazin-1-yl; or —X′—C_{1, 2 or 3}-alkylene-NR₁₉R₂₀ wherein X′ is a direct bond, O or NH;
12. In the residue of formula (a) either each of R₂ and R₃ is H or one of R₂ and R₃ is H and the other is F, Cl, CH₃, OH, OCH₃ or CF₃;
13. In the residue of formula (a) R₂ is OH;
14. In the residue of formula (b) either each of R₅ and R₆ is H or one of R₅ and R₆ is H and the other is F, Cl, CH₃, OCH₃ or CF₃;
15. In the residue of formula (b) R₄ is a radical of formula (α) or NR₁₆R₁₇;
16. In the residue of formula (d) either each of R₉ and R₁₀ is H or one of R₉ and R₁₀ is H and the other is F, Cl, CH₃, OCH₃ or CF₃; preferably R₁₀ is H and R₉ is in position 5, 6, 7 or 8, preferably in position 6;
17. In the residue of formula (d), each of R₉ and R₁₀ is H, R₈ is optionally substituted piperazin, e.g. R₈ is 4-methyl-piperazin-1-yl
18. In the residue of formula (e) each of R₁₂ and R₁₃ is H;
19. In the residue of formula (e) one of R₁₂ and R₁₃ is H and the other is F, Cl, CH₃, OCH₃ or CF₃;
   when E is —N═ and G is —CH═, preferably R₁₃ is H and R₁₂ is in position 6 or 7;
   when E is —H═ and G is —N═, preferably R₁₃ is H and R₁₂ is in position 7;
20. In the residue of formula (e), each of R₁₂ and R₁₃ is H; E is CH═ and G is —N═, R₁₁ is 4,7-diaza-spiro[2.5]oct-7-yl; or piperazin-1-yl substituted in position 3 by methyl or ethyl and optionally in position 4 by methyl,
21. In the residue of formula (f) R₁₅ is H, CH₃ or Cl, e.g. in position 5 or 6;
22. In the residue of formula (f) R′₁₅ is H or CH₃, e.g. in position 5, preferably H;
23. R is a radical of formula (d), (e) or (f).

In the compounds of formula III, the following significances are preferred:

each of R₄₄, R′₄₄, R₄₅, R′₄₅, R₄₆, R′₄₆, R₄₇ and R′₄₇ is hydrogen;

R₄₁ is

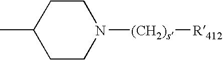

wherein either s′ is 0 and R′₁₂ is hydrogen or C₁₋₄alkyl; or s′ is 1 and R′₄₁₂ is pyridyl, preferably 2-pyridyl, and

R_{41′} is H; or C₁₋₄alkyl.

Preferred compounds of formula II are 3-(1.H.-indol-3-yl)-4-[2-(4-methyl-piperazin-1-yl)-quinazolin-4-yl]-pyrrole-2,5-dione (referred to hereinafter as Compound A), 3-(1.H.-indol-3-yl)-4-[2-(piperazin-1-yl)-quinazolin-4-yl]-pyrrole-2,5-dione (referred to hereinafter as Compound B), 3-[3-(4,7-Diaza-spiro[2.5]oct-7-yl)-isoquinolin-1-yl]-4-(7-methyl-1H-indol-3-yl)-pyrrole-2,5-dione (Compound C), in free form or in a pharmaceutically acceptable salt form, e.g. the acetate salt thereof.

Preferred compounds of formula II are 3-(1-methyl-1H-indol-3-yl)-4-[1-{(1-pyridin-2-ylmethyl)-piperidin-4-yl}-1H-indol-3-yl]-pyrrole-2,5-dione (Compound D), 3-(1-methyl-1H-indol-3-yl)-4-[1-(piperidin-4-yl)-1H-indol-3-yl]-pyrrole-2,5-dione (Compound E), or a pharmaceutically acceptable salt, hydrate or solvate thereof.

JAK3 is an enzyme which is primarily expressed in T and B cells and plays a critical role in T cell development and function. JAK3 kinase inhibitors are e.g. compounds having an IC₅₀ value <5 μM, preferably <1 μM, more preferably <0.1 μM in the following assays:

Interleukin-2 (IL-2) Dependent Proliferation Assays with CTL/L and HT-2 Cells

The IL-2 dependent mouse T cell lines CTL/L and HT-2 are cultured in RPMI 1640 (Gibco 52400-025) supplemented with 10% Fetal Clone I (HyClone), 50 μM 2-mercaptoethanol (31350-010), 50 μg/ml gentamycine (Gibco 15750-037), 1 mM sodium pyruvate (Gibco 11360-039), non-essential amino acids (Gibco 11140-035; 100×) and 250 U/ml mouse IL-2 (supernatant of X63-Ag8 transfected cells containing 50'000 U/ml mouse IL-2 according to Genzyme standard). Cultures are split twice a week 1:40.

Before use the cells are washed twice with culture medium without mouse IL-2. The proliferation assay is performed with 4000 CTL/L cells/well or 2500 HT-2 cells/well in flat-bottom 96-well tissue culture plates containing appropriate dilutions of test compounds in culture medium with 50 U/ml mouse IL-2. CTL/L cultures are incubated at 37° C. for 24 h and HT-2 cultures are incubated for 48 h. After addition of 1 μCi ³H-thymidine and a further overnight incubation cells are harvested onto fibre filters and radioactivity is counted.

### Interleukin-2 Dependent Proliferation of Human Peripheral Blood Mononuclear Cells

Human peripheral blood mononuclear cells are isolated on Ficoll from buffy coats with unknown HLA type (Blutspendezentrum, Kantonsspital, Basel, Switzerland). Cells are kept at 2×10⁷ cells/ml (90% FCS, 10% DMSO) in cryotubes (Nunc) in liquid nitrogen until use.

The cells are incubated for four days at 37° C. in a humidified CO₂ (7%) incubator in costar flasks at the concentration of 7×10⁵ cells/ml in culture medium containing RPMI 1640 (Gibco, Pacely, England) supplemented with Na-pyruvate (1 mM; Gibco), MEM nonessential amino acids and vitamins (Gibco), 2-mercaptoethanol (50 μM), L-glutamine (2 mM), gentamicin and penicillin/streptomycin (100 μg/ml; Gibco), bacto asparagine (20 □g/ml; Difco), human insulin (5 □g/ml; Sigma), human transferrin (40 □g/ml; Sigma), selected fetal calf serum (10%, Hyclone Laboratories, Logan, Utah) and 100 μg/ml phytohemagglutinine. Cells are washed twice in RPMI 1640 medium containing 10% FCS and incubated for 2 hours. After centrifugation the cells are taken up in the culture medium mentioned above (without phytohemagglutinine) containing interleukin-2 (Chiron 200 U/ml), distributed in triplicates into flat-bottomed 96-well tissue culture plates (Costar #3596) at a concentration of 5×10⁴ cells/0.2 ml in the presence of appropriate concentrations of test compounds and incubated at 37° C. for 72 hours. ³H-thymidine (1 μCi/0.2 ml) was added for the last 16 hours of culture. Subsequently cells are harvested and counted on a scintillation counter.

Suitable JAK3 kinase inhibitors include e.g.
Compounds as disclosed in USP 2003/0073719A1, e.g. a compound of formula IV

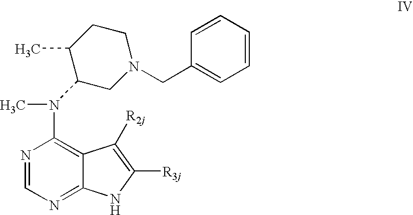

wherein

each of R₂ⱼ and R₃ⱼ independently is selected from the group consisting of H, amino, halogen, OH, nitro, carboxy, C₂₋₆alkenyl, C₂₋₆alkynyl, CF₃, trifluoromethoxy, C₁₋₆alkyl, C₁₋₆alkoxy, C₃₋₆cycloalkyl wherein the alkyl, alkoxy or cycloalkyl groups are optionally substituted by one to three groups selected from halogen, OH, carboxy, amino, C₁₋₆alkylthio, C₁₋₆ alkylamino, (C₁₋₆alkyl)₂amino, C₅₋₉heteroaryl, C₂₋₉heterocycloalkyl, C₃₋₉cycloalkyl or C₆₋₁₀aryl; or each of R₂ⱼ and R₃ⱼ independently is C₃₋₁₀cycloalkyl, C₃₋₁₀cycloalkoxy, C₁₋₆alkylamino, (C₁₋₆ alkyl)₂amino, C₆₋₁₀arylamino, C₁₋₆alkylthio, C₆₋₁₀arylthio, C₁₋₆alkylsulfinyl, C₆₋₁₀arylsulfinyl, C₁₋₄alkylsulfonyl, C₆₋₁₀arylsulfonyl, C₁₋₆acyl, C₁₋₆alkoxy-CO—NH—, C₁₋₆alkylamino-CO—, C₅₋₉heteroaryl, C₂₋₉ heterocycloalkyl or C₆₋₁₀aryl wherein the heteroaryl, heterocycloalkyl and aryl groups are optionally substituted by one to three halogeno, C₁₋₆alkyl, C₁₋₆alkyl-CO—NH—, C₁₋₆alkoxy-CO—NH—, C₁₋₆alkyl-CO—NH—C₁₋₆alkyl, C₁₋₆alkoxy-CO—NH—C₁₋₆alkyl, C₁₋₆alkoxy-CO—NH—C₁₋₆ alkoxy, carboxy, carboxy-C₁₋₆alkyl, carboxy-C₁₋₆alkoxy, benzyloxycarbonyl-C₁₋₆alkoxy, C₁₋₆ alkoxycarbonyl-C₁₋₆alkoxy, C₆₋₁₀aryl, amino, aminoC₁₋₆alkyl, C₂₋₇alkoxycarbonylamino, C₆₋₁₀ aryl-C₂₋₇alkoxycarbonylamino, C₁₋₆alkylamino, (C₁₋₆alkyl)₂amino, C₁₋₆alkylamino-C₁₋₆alkyl, (C₁₋₆alkyl)₂amino-C₁₋₆alkyl, hydroxy, C₁₋₆alkoxy, carboxy, carboxy-C₁₋₆alkyl, C₂₋₇alkoxycarbonyl, C₂₋₇alkoxycarbonyl-C₁₋₆alkyl, C₁₋₆alkoxy-CO—NH—, C₁₋₆alkyl-CO—NH—, cyano, C₅₋₉ heterocycloalkyl, amino-CO—NH—, C₁₋₆alkylamino-CO—NH—, (C₁₋₆alkyl)₂amino-CO—NH—, C₆₋₁₀ arylamino-CO—NH—, C₅₋₉heteroarylamino-CO—NH—, C₁₋₆alkylamino-CO—NH—C₁₋₆alkyl, (C₁₋₆ alkyl)₂amino-CO—NH—C₁₋₆alkyl, C₁₋₁₀arylamino-CO—NH—C₁₋₆alkyl, C₅₋₉heteroarylamino-CO—NH—C₁₋₆-alkyl, C₁₋₆alkylsulfonyl, C₁₋₆alkylsulfonylamino, C₁₋₆alkylsulfonylaminoC₁₋₆alkyl, C₆₋₁₀ arylsulfonyl, C₆₋₁₀arylsulfonylamino, C₆₋₁₀arylsulfonylamino-C₁₋₆-alkyl, C₁₋₆alkylsulfonylamino, C₁₋₆alkylsulfonylamino-C₁₋₆alkyl, C₅₋₉heteroaryl or C₂₋₉heterocycloalkyl; for example methyl-[(3R,4R)-4-methyl-1-(propane-1-sulfonyl)-piperidin-3-yl]-(7H-pyrrolo[2,3-d]pyrimidin-4-yl)-amine; (3R,4R)—)-4-methyl-3-[methyl-(7H-pyrrolo[2-,3-d]pyrimidin-4-yl)-amino]-piperidine-1-carboxylic acid methyl ester; 3,3,3-trifluoro-1-{(3R,4R)-4-methyl-3-[methyl-(7H-pyrrolo[2,3-d]pyrimidin-4-yl)-amino]-piperidin-1-yl}-propan-1-one; (3R,4R)-4-methyl-3-[methyl-(7H-pyrrolo[2,3-d]pyrimidin-4-yl)-amino]-piperidine-1-carboxylic acid dimethylamide; {(3R,4R)-4-methyl-3-[methyl-(7H-pyrrolo[2,3-d]pyrimidin-4-yl)-amino]-piperidine-1-carbonyl}amino)-acetic acid ethyl ester; 3{(3R,4R)-4-Methyl-3-[methyl-(7H-pyrrolo[2,3-d]pyrimidin-4-yl)-amino]-piperidin-1-yl}-3-oxo-propionitrile; 3,3,3-trifluoro-1-{(3R,4R)-4-methyl-3-[-methyl-(5-methyl-7H-pyrrolo[2,3-d]pyrimidin-4-yl)-amino]-piperidin-1-yl}-propan-1-one; 1-{(3R,4R)-4-methyl-3-[methyl-(7H-pyrrolo[2,3-d]pyrimidin-4-y-l)-amino]piperidin-1-yl}-but-3-yn-1-one; 1-{(3R,4R)-3-[(5-chloro-7H-pyrrol-o[2,3-d]pyrimidin-4-yl)-methyl-amino]-4-methyl-piperidin-1-yl}-propan-1-one; 1-{(3R,4R)-3-[(5-fluoro-7H-pyrrolo[2,3-d]pyrimidin-4-yl)-methyl-amino]—4-methyl-piperidin-1-yl}-propan-1-one; (3R,4R)—N-cyano-4-methyl-3-[methyl-(7H-pyrrolo[2,3-d]pyrimidin-4-yl)-amino]-N′-propyl-piperidine-1-carboxamidine; or (3R,4R)—N-cyano-4, N′,N′-trimethyl-3-[methyl-(7H-pyrrolo[2,3-d]pyrimidin-4-yl)-amino]-piperidine-1-carboxamidine;
Compounds as disclosed in WO 01/042246, e.g. a compound of formula IVb

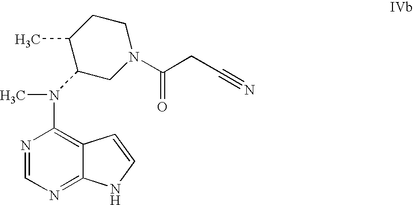

Compounds as disclosed in WO 02/092571, e.g. a compound of formula V

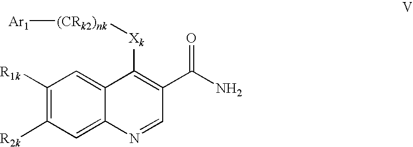

wherein

Ar₁ is selected from phenyl, tetrahydronaphthenyl, indolyl, pyrazolyl, dihydroindenyl, 1-oxo-2,3-dihydroindenyl or indazolyl, each of which can be optionally substituted by one or more groups selected from halogen, hydroxy, cyano, C₁₋₈alkoxy, CO₂R₈ₖ, CONR₉ₖR₁₀ₖ, C₁₋₈alkyl-O—C₁₋₈alkyl, C₁₋₈alkyl-NR₈ₖ—C₁₋₈alkyl, C₁₋₈alkyl-CONR₈—C₁₋₈alkyl, C₁₋₈alkyl-CONR₉ₖR₁₀ₖ, NR₈ₖCOC₁₋₈alkyl, C₁₋₈thioalkyl, C₁₋₈alkyl (itself optionally substituted by one or more OH or cyano or fluorine) or C₁₋₈alkoxy;

Xₖ is NR₃ₖ or O; nₖ is 0 or 1;

each Rₖ group independently is hydrogen or C₁₋₈alkyl;

each of R₁ₖ and R₂ₖ independently is selected from H, halogen, nitro, cyano, C₁₋₈alkyl, C₁₋₈alkoxy, OH, aryl, Yₖ(CR₁₁ₖ₂)ₚₖNR₄ₖR₅ₖ, Yₖ(CR₁₁ₖ₂)ₚₖCONR₄ₖR₅ₖYₖ(CR₁₁ₖ₂)ₚₖCO₂R₆ₖ, Yₖ(CR₁₁ₖ₂)ₚₖOR₆ₖ; Yₖ(C R₁₁ₖ₂)ₚₖ R₆ₖ; or R₁ₖ and R₂ₖ are linked together as —OCHO— or —OCH₂CH₂O—;

each R₁₁ₖ independently is H, C₁₋₈alkyl, hydroxy or halogen; pₖ is 0, 1, 2, 3, 4 or 5;

R₃ₖ is H or C₁₋₈alkyl;

Yₖ is oxygen, CH₂ or NR₇ₖR₃ₖ is hydrogen or C₁₋₈alkyl;

each of R₄ₖ and R₅ₖ independently is H, C₁₋₈alkyl or R₄ₖ and R₅ₖ together with the nitrogen atom to which they are attached form a 4-to 7-membered saturated or aromatic heterocyclic ring system optionally containing a further O, S or NR₆ₖ, or one of R₄ₖ and R₅ₖ is H or C₁₋₈ alkyl and the other is a 5- or 6-membered heterocyclic ring system optionally containing a further O, S or N atom;

R₆ₖ is H, C₁₋₈alkyl, phenyl or benzyl;

R₇ₖ is H or C₁₋₈alkyl;

R₈ₖ is H or C₁₋₈alkyl; each of R₉ₖ and R₁₀ independently is hydrogen or C₁₋₈alkyl;
Compounds as disclosed in US 2002/0055514A1, e.g. a compound of formula VI

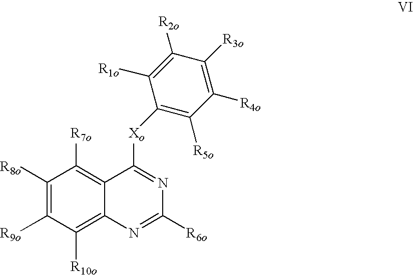

wherein

Xₒ is NH, NR₁₁ₒ, S, O CH₂ or R₁₁ₒCH;

R₁₁ₒ is H, C₁₋₄alkyl or C₁₋₄alkanoyl;

each of R₁ₒ to R₈ₒ, independently, is H, halogen, OH, mercapto, amino, nitro, C₁₋₄alkyl, C₁₋₄alkoxy or C₁₋₄alkylthio; wherein 2 of R₁ₒ—R₅ₒ together with the phenyl ring to which they are attached may optionally form a fused ring, for example, forming a naphthyl or a tetrahydronaphthyl ring; and further wherein the ring formed by the two adjacent groups of R₁ₒ—R₅ₒ may optionally be substituted by 1, 2, 3 or 4 halogen, hydroxy, mercapto, amino, nitro, C₁₋₄alkyl, C₁₋₄alkoxy or C₁₋₄alkylthio; and provided that at least one of R₂ₒ—R₅ₒ is OH, and

each of R₉ₒ and R₁₀ₒ independently is H, halogen, C₁₋₄alkyl, C₁₋₄alkoxy or C₁₋₄alkanoyl; or R₉ₒ and R₁₀ₒ together are methylenedioxy;
Compounds as disclosed in WO 04/052359, e.g. a compound of formula VII

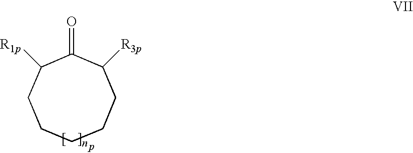

wherein nₚ is 1, 2, 3, 4 or 5;

R₁ₚ is H, CH₃ or CH₂N(CH₃)₂; and

R₃ₚ is CH₂N(CH₃)₂

in free form or in a pharmaceutically acceptable salt form.

The compounds of formulae IV to VII may exist in free or salt form. Examples of pharmaceutically acceptable salts of the compounds of the formulae IV to VI include salts with inorganic acids, such as hydrochloride, salts with organic acids, such as acetate or citric acid, or, when appropriate, salts with metals such as sodium or potassium, salts with amines, such as triethylamine and salts with dibasic amino acids, such as lysine.

When the compounds of formulae IV to VII have one or more asymmetric centers in the molecule, the present invention is to be understood as embracing the various optical isomers, as well as racemates, diastereoisomers and mixtures thereof are embraced. When the compounds of formulae IV to VII comprise a double bond, the compounds may exist as cis or trans configurations or as mixtures thereof.

In formula IV C₆₋₁₀aryl is phenyl or naphthyl. C₂₋₉heterocycloalkyl may be e.g. pyrrolidinyl, tetrahydrofuranyl, dihydrofuranyl, tetrahydropyranyl, pyranyl, thiopyranyl, aziridinyl, oxiranyl, methylenedioxy, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, morpholinyl, piperazinyl, etc. Such a group will be attached either by a C or N atom. C₂₋₉heteroaryl may be e.g. furyl, thienyl, thiazolyl, pyrazolyl, isothizolyl, oxazolyl, isoxazolyl, pyrrolyl, triazolyl, tetrazolyl, imidazolyl, oxadiazolyl, thiadiazolyl, pyridyl, pyrimidyl, pyrazinyl, pyridazinyl, triazinyl, pyrazolo[3,4b]pyridinyl, cinnolinyl, pteridinyl, purinyl, benzoxazolyl, benzothiazolyl, benzofuranyl, isoindolyl, indolyl, indolizinyl, indazolyl, isoquinolyl, quinolyl, phthalazinyl, quinoxalinyl, quinazolinyl, benzoxazinyl, etc. Such a group will be attached either by a C or N atom.

Preferred JAK3 kinase inhibitors include e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)amino-6,7-dimethoxyquinazoline] (WHI-P154), [4-(3′,5′-dibromo-4′-hydroxylphenyl)-amino-6,7-dimethoxyquinazoline] WHI-P97, and 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate, or a compound of formula IVb, in free form or in a pharmaceutically acceptable salt form, e.g. mono-citrate (also called CP-690,550) or a compound of formula VII.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e. a pharmaceutical combination within the scope of this invention could include three active ingredients or more.

In accordance with the particular findings of the present invention, there is provided

1. A pharmaceutical combination comprising:
a) at least one PKC inhibitor, and
b) at least one JAK3 kinase inhibitor.
   2. A method for treating or preventing an autoimmune disease or disorder, or cell, tissue or organ graft rejection in a subject in need thereof, comprising co-administration to said subject, e.g. concomitantly or in sequence, of a therapeutically effective amount of at least one PKC inhibitor, and preferably at least one JAK3 kinase inhibitor, e.g. as disclosed above. Examples of autoimmune diseases include e.g. sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, allergic rheumatoid arthritis, systemic lupus erythematosus, nephrotic syndrome lupus, Hashimoto's thyroiditis, multiple sclerosis, myasthenia gravis, type I diabetes mellitus and complications associated therewith, type II adult onset diabetes mellitus, uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia areata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, keratoleukoma, ocular pemphigus, Mooren's ulcer, scleritis, Graves' opthalmopathy, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease and ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), B-virus hepatitis, non-A/non-B hepatitis and cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, amyotrophic lateral sclerosis or rheumatic fever.

By graft rejection is meant acute or chronic rejection of cells, tissue or solid organ allo- or xenografts of e.g. pancreatic islets, stem cells, bone marrow, skin, muscle, corneal tissue, neuronal tissue, heart, lung, combined heart-lung, kidney, liver, bowel, pancreas, trachea or oesophagus, or graft-versus-host diseases. Chronic rejection may also be named graft vessel diseases or graft vasculopathies.

3. A pharmaceutical combination as defined under 1) above, e.g. in the form of a kit which may further comprise instructions for the administration, e.g. for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, for example in accordance with the methods hereinafter described.

### A. Rat Heart Transplantation

The strain combination used: Male Lewis (RT¹ haplotype) and BN (RT¹ haplotype). The animals are anaesthetised using inhalational isofluorane. Following heparinisation of the donor rat through the abdominal inferior vena cava with simultaneous exsanguination via the aorta, the chest is opened and the heart rapidly cooled. The aorta is ligated and divided distal to the first branch and the brachiocephalic trunk is divided at the first bifurcation. The left pulmonary artery is ligated and divided and the right side divided but left open. All other vessels are dissected free, ligated and divided and the donor heart is removed into iced saline.

The recipient is prepared by dissection and cross-clamping of the infra-renal abdominal aorta and vena cava. The graft is implanted with end-to-side anastomoses, using 10/0 monofilament suture, between the donor brachiocephalic trunk and the recipient aorta and the donor right pulmonary artery to the recipient vena cava. The clamps are removed, the graft tethered retroabdominally, the abdominal contents washed with warm saline and the animal is closed and allowed to recover under a heating lamp. Graft survival is monitored by daily palpation of the beating donor heart through the abdominal wall. Rejection is considered to be complete when heart beat stops. Increases of graft survival are obtained in animals treated with a combination according to the invention, e.g. a combination of Compound A, e.g. in acetate salt form, and the compound CP-690,550 in the mono-citrate salt form, each component of the combination being administered orally at a daily dose of 0.1 to 50 mg/kg. Thus Compound A in the acetate form, when administered at a dose of 1 to 30 mg/kg/day, and CP-690,550 mono-citrate, when administered at an EC₅₀ (drug concentration in blood at which 50% of the animals maintain their graft for >28 days) of 60 ng/ml, significantly increase the graft survival.

### B. Combined Treatment

Suitable clinical studies are, for example, open label, dose escalation studies in patients with psoriasis or multiple sclerosis. Such studies prove in particular the synergism of the active ingredients of the combination of the invention. The beneficial effects on psoriasis or multiple sclerosis can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

Alternatively, a placebo-controlled, double blind study can be used in order to prove the benefits of the combination of the invention mentioned herein, e.g. in transplantation of an organ, tissue or cells, e.g. Langerhans islet cells.

The administration of a pharmaceutical combination of the invention results not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective against graft rejection or autoimmune diseases or disorders associated therewith comprising a combination of the invention. In this composition, agent a) and agent (b) may be administered together, one after the other or separately in one combined unit dosage form or in two separate unit dosage forms. The unit dosage form may also be a fixed combination.

The pharmaceutical compositions for separate administration of agent a) and agent b) or for the administration in a fixed combination, i.e. a single galenical composition comprising at least two combination partners a) and b), according to the invention may be prepared in a manner known per se and are those suitable for enteral, e.g. oral, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating graft rejection or autoimmune diseases according to the invention may comprise (i) administration of the first agent a) in free or pharmaceutically acceptable salt form and (ii) administration of an agent b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A physician, clinician or veterinarian of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent a) or b) or will, of course, vary depending on a variety of factors, for example the compound chosen, the particular condition to be treated and the desired effect. In general, however, satisfactory results are achieved on administration of agent a) at daily dosage rates of the order of about 0.1 to about 100 mg/kg per day, as a single dose or in divided doses. The PKC inhibitor, e.g. a compound of formulae I to III, e.g. Compound A, B, C, D or E, may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. An indicated daily dosage for oral administration in the larger mammal, e.g. humans, is in the range from about 0.5 mg to about 2000 mg active ingredient, e.g. Compound A, B, C, D or E, conveniently administered, for example, in divided doses up to four times a day or in retard form.

Agent b), e.g. CP-690,550 or a compound of formula XVII, may be administered to a human in a daily dosage range of 0.5 to 1000 mg. Suitable unit dosage forms for oral administration comprise from ca. 0.1 to 500 mg active ingredient, together with one or more pharmaceutically acceptable diluents or carriers therefor.

The administration of a pharmaceutical combination of the invention results not only in a beneficial effect, e.g. a synergistic therapeutic effect, e.g. with regard to inhibiting graft rejection in transplanted patients or slowing down or arresting autoimmune disorders, but also in further surprising beneficial effects, e.g. less side-effects, an improved quality of life or a decreased morbidity, compared to a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

A preferred combination is the combination of compound A, B, C, D or E, preferably compound A, even more preferably compound A in form of acetate salt, with CP-690,555 monocitrate.

1. A pharmaceutical combination comprising:
a) at least one PKC inhibitor, and
b) at least one JAK3 kinase inhibitor.

2. The pharmaceutical combination according to claim 1 wherein agent a) is a PKC inhibitor selected from a compound of formulae I and II as hereinabove described, a pharmaceutically acceptable salt or hydrate thereof, a compound of formula III as hereinabove described, a pharmaceutically acceptable salt, hydrate or solvate thereof.

3. The pharmaceutical combination according to claim 1 wherein agent b) is a JAK3 kinase inhibitor having an IC₅₀ value <5 μM in the IL-2 dependent proliferation assay with CTL/L and HT-2 cells and in the IL-2 dependent proliferation assay of human peripheral blood mononuclear cells.

4. The pharmaceutical combination according to claim 1 wherein agent b) is selected from a compound of formulae IV to VII as hereinabove described, or a pharmaceutically acceptable salt thereof.

5. The pharmaceutical combination according to claim 1 wherein the agent a) is 3-(1.H.-indol-3-yl)-4-[2-(4-methyl-piperazin-1-yl)-quinazolin-4-yl]-pyrrole-2,5-dione; 3-(7.H.-indol-3-yl)-4-[2-(piperazin-1-yl)-quinazolin-4-yl]-pyrrole-2,5-dione; or 3-[3-(4,7-Diaza-spiro[2.5]oct-7-yl)-isoquinolin-1-yl]-4-(7-methyl-1H-indol-3-yl)-pyrrole-2,5-dione, in free form or in a pharmaceutically acceptable salt form or in hydrate form; 3-(1-methyl-1H-indol-3-yl)-4-[1-{(1-pyridin-2-ylmethyl)-piperidin-4-yl}-1H-indol-3-yl]-pyrrole-2,5-dione or 3-(1-methyl-1H-indol-3-yl)-4-[1-(piperidin-4-yl)-1H-indol-3-yl]-pyrrole-2,5-dione, or a pharmaceutically acceptable salt, hydrate or solvate thereof; preferably wherein the agent a) is the acetate salt of 3-(7.H.-indol-3-yl)-4-[2-(4-methyl-piperazin-1-yl)-quinazolin-4-yl]-pyrrole-2,5-dione.

6. The pharmaceutical combination according to claim 1 wherein the JAK3 kinase inhibitor b) is 3-{(3R,4R)-4-methyl-3-[methyl-(7H-pyrrolo[2,3-d]pyrimidin-4-yl)-amino]-piperidin-1-yl}-3-oxo-propionitrile or a compound or formula XVII as defined in claim 5, in free form or in a pharmaceutically acceptable salt form.

7. The pharmaceutical combination according to claim 1 wherein the JAK3 kinase inhibitor b) is CP-690,550 in free form or in a pharmaceutically acceptable salt form.

8-9. (canceled)

10. A method for treating or preventing an autoimmune disease or disorder, or cell, tissue or organ graft rejection in a subject in need thereof, comprising co-administration to said subject a therapeutically effective amount of at least one PKC inhibitor and at least one JAK3 kinase inhibitor.
